(19) United States
(12) Patent Application Publication
(10) Pub. No.: US 2015/0018677 A1
(43) Pub. Date: Jan. 15, 2015

(54) ULTRASOUND DIAGNOSIS APPARATUS AND CONTROL METHOD

(71) Applicants: Kabushiki Kaisha Toshiba, Minato-ku (JP); Toshiba Medical Systems Corporation, Otawara-shi (JP)

(72) Inventors: Hiroki YOSHIARA, Nasushiobara-shi (JP); Makoto Hirama, Otawara-shi (JP); Tetsuya Yoshida, Bergschenhoek (NL); Akihiro Kakee, Nasushiobara-shi (JP); Tetsuya Kawagishi, Nasushiobara-shi (JP); Hiroyuki Shikata, Nasushiobara-shi (JP)

(73) Assignees: Kabushiki Kaisha Toshiba, Minato-ku (JP); Toshiba Medical Systems Corporation, Otawara-shi (JP)

(21) Appl. No.: 14/327,736

(22) Filed: Jul. 10, 2014

(30) Foreign Application Priority Data
Jul. 12, 2013 (JP) 2013-146964

(51) Int. Cl. A61B 8/08; A61B 8/00; A61B 8/14

(52) U.S. Cl. CPC A61B 8/5207; A61B 8/14; A61B 8/54
USPC 600/431; 600/447

(57) ABSTRACT

An ultrasound diagnosis apparatus includes a transmitter/receiver, a signal processor, and an image generator. The transmitter/receiver transmits, at least once in each scanning line, a first ultrasound pulse and a second ultrasound pulse whose amplitude corresponds to amplitude of the first ultrasound pulse modulated at a given ratio and acquires a received-signal group consisting of multiple received signals. The signal processor acquires, on the basis of a coefficient that reduces the energy of a first composite signal that is obtained by combining, in accordance with the given ratio, multiple received signals contained in the first received signal group acquired by the transmitter/receiver, a second composite signal by combining multiple received signals contained in a second received signal group that is acquired by the transmitter/receiver and that is different from the first received signal group. The image generator generates an ultrasound image based on the second composite signal.

### CROSS-REFERENCE TO RELATED APPLICATIONS

This application is based upon and claims the benefit of priority from Japanese Patent Application No. 2013-146964, filed on Jul. 12, 2013; the entire contents of which are incorporated herein by reference.

### FIELD

Embodiments described herein relate generally to an ultrasound diagnosis apparatus and a control method.

### BACKGROUND

In recent years, intravenous contrast agents have been made into commercial products and a contrast echo method known as contrast harmonic imaging (CHI) is now performed using ultrasound diagnosis apparatuses. The aim of the contrast echo method is, during an examination of, for example, the heart or liver, to enhance the blood flow signals by injecting an ultrasound contrast agent intravenously and then to evaluate the hemodynamics. In many ultrasound contrast agents, microbubbles function as the reflection source. However, due to the delicate nature of the microbubble base, the microbubbles are broken down due to the mechanical effect of the ultrasound even when the ultrasound exposure is at the level of a normal diagnosis, which results in a decrease in the intensity of signals from the scanned surface.

Accordingly, in order to observe the dynamics of reflux in real time, it is necessary to make a relative reduction in the breaking down of microbubbles due to scanning by, for example, performing imaging by transmitting ultrasound at a low sound pressure. Such imaging from low sound-pressure ultrasound transmission also reduces the signal/noise ratio (S/N ratio). In order to compensate for this, various signal processing methods, such as phase modulation (PM), amplitude modulation (AM) and amplitude and phase modulation (AMPM), have been devised. Such visualization methods allow contrast-enhanced images with a higher S/N ratio to be displayed in real time. Ultrasound contrast agents are, because they offer performance in real time and high spatial resolution, used to carefully examine minute structures (such as microvessel structures) that cannot be visualized with X-ray CT apparatus or MRI apparatus.

For example, amplitude modulation is a visualization method that offers an excellent bubble-tissue ratio and tremendous depth sensitivity. Amplitude modulation is a visualization method where, while non-linear responses from a contrast agent are extracted, linear signals from body tissue are canceled out in order for specific extraction of the contrast agent. Accurate waveform formation is required to implement amplitude modulation. However, depending on how the ultrasound diagnosis apparatus is implemented (e.g., the system configuration, the aperture control and amplitude control) and the effect of nonlinearity of the circuit, an ultrasound diagnosis apparatus is not necessarily able to completely cancel out linear signals derived from tissue. In such a case, tissue-derived linear signals remain, which reduces the bubble-tissue ratio in the contrast-enhanced images.

### BRIEF DESCRIPTION OF THE DRAWINGS

FIG. 1 is a block diagram of an exemplary configuration of an ultrasound diagnosis apparatus according to a first embodiment;

FIG. 2 is a block diagram of an exemplary configuration of a B-mode processing unit according to the first embodiment;

FIG. 3A illustrates filter coefficients according to the first embodiment;

FIG. 3B illustrates amplitude and phase properties of the filter according to the first embodiment;

FIG. 4 illustrates filter coefficient designing processing performed by a filter coefficient designing unit according to the first embodiment;

FIG. 5 illustrates generated image based on the filter coefficient by the filter coefficient designing unit according to the first embodiment;

FIG. 6 is a flowchart of a procedure of processing performed by an ultrasound diagnosis apparatus according to the first embodiment;

FIG. 7 is a flowchart of a procedure of ultrasound transmitting/receiving processing performed by the ultrasound diagnosis apparatus according to the first embodiment;

FIG. 8 is a flowchart of a procedure of B-mode data generation processing performed by the ultrasound diagnosis apparatus according to the first embodiment;

FIG. 9 is a block diagram of an exemplary configuration of a B-mode processing unit according to a second embodiment;

FIG. 10 is a flowchart of a procedure of processing performed by an ultrasound diagnosis apparatus according to the second embodiment;

FIG. 11 is a flowchart of a procedure of filter designing processing performed by the ultrasound diagnosis apparatus according to the second embodiment; and

FIG. 12 is a flowchart of a procedure of processing performed by an ultrasound diagnosis apparatus according to a third embodiment.

### DETAILED DESCRIPTION

An ultrasound diagnosis apparatus according to an embodiment includes a transmitter/receiver, a signal processor, and an image generator. The transmitter/receiver transmits, at least once in each scanning line, a first ultrasound pulse and a second ultrasound pulse, whose amplitude is equivalent to amplitude which is acquired by modulating amplitude of the first ultrasound pulse by using a given ratio, and acquires a received-signal group consisting of multiple received signals based on the transmitting. The signal processor acquires, on the basis of a coefficient that reduces the energy of a first composite signal that is obtained by combining, in accordance with the given ratio, multiple received signals contained in a first received signal group acquired by the transmitter/receiver, a second composite signal by combining multiple received signals contained in a second received signal group that is acquired by the transmitter/receiver and that is different from the first received signal group. The image generator generates an ultrasound image based on the second composite signal.

### First Embodiment

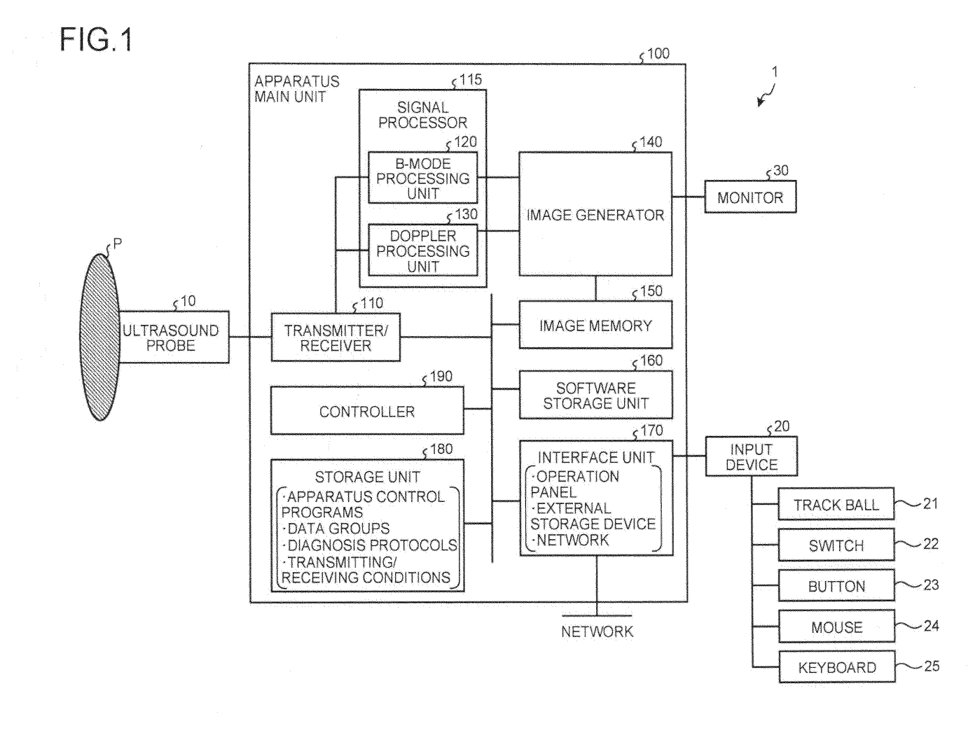

The configuration of the ultrasound diagnosis apparatus according to a first embodiment will be described with FIG. 1. FIG. 1 is a block diagram of an exemplary configuration of an ultrasound diagnosis apparatus 1 according to the first embodiment. As illustrated in FIG. 1, the ultrasound diagnosis apparatus 1 includes an ultrasound probe 10, an input device 20, a monitor 30, and an apparatus main unit 100.

The ultrasound probe 10 includes multiple piezoelectric transducer elements that generate ultrasound according to a drive signal that is supplied from a transmitter/receiver 110 of the apparatus main unit 100, which will be described below. The ultrasound probe 10 receives reflected-wave signals from a patient P and converts the reflected-wave signals into electrical signals. The ultrasound probe 10 includes a matching layer that is provided to the piezoelectric transducer elements and a backing member that prevents backward propagation of ultrasound from the piezoelectric transducer elements. The ultrasound probe 10 is detachably connected to the apparatus main unit 100.

When ultrasound is transmitted from the ultrasound probe 10 to the patient P, the transmitted ultrasound is sequentially reflected on a surface of a body tissue of the patient P where acoustic impedance discontinuity occurs and is received as reflected-wave signals by the multiple piezoelectric transducer elements of the ultrasound probe 10. The amplitude of the received reflected-wave signals depends on the difference in acoustic impedance on the discontinuity surface on which ultrasound is reflected. The reflected-wave signals obtained when the transmitted ultrasound pulses are reflected on the flowing blood or the surface of the heart wall have a frequency shift due to the Doppler effect depending on the velocity components of the moving object in the direction in which ultrasound is transmitted.

The input device 20 is connected to the apparatus main unit 100 and includes a track ball 21, various switches 22, various buttons 23, a mouse 24, and a keyboard 25. The input device 20 notifies the apparatus main unit 100 of various instructions from an operator. For example, various instructions include an instruction for setting a region of interest (ROI), an instruction for setting imaging conditions including an ultrasound transmission condition, and an instruction for displaying the time elapsing from administration of a contrast agent to the patient P.

The monitor 30 displays a graphical user interface (GUI) for the operator of the ultrasound diagnosis apparatus 1 to make various settings by using the input device 20 and displays ultrasound images that are generated by the apparatus main unit 100. Specifically, the monitor 30 displays in-vivo morphological information and blood flow information as images on the basis of video signals that are input from an image generator 140, which will be described below. Upon receiving an instruction for displaying the time elapsing from administration of the contrast agent to the patient P, the monitor 30 displays the time after the administration of the contrast agent.

The apparatus main unit 100 generates an ultrasound image on the basis of the reflected-wave signals that are received by the ultrasound probe 10. As illustrated in FIG. 1, the apparatus main unit 100 includes a transmitter/receiver 110, a signal processor 115, the image generator 140, an image memory 150, a software storage unit 160, an interface unit 170, a storage unit 180, and a controller 190. The transmitter/receiver 110, a B-mode processing unit 120, a Doppler processing unit 130, the image generator 140 etc. may be implemented with hardware, such as an integrated circuit, or may implemented with a module software program.

The transmitter/receiver 110 includes a delay circuit, a pulser circuit, and a trigger generation circuit, which are not shown, and supplies a drive signal to the ultrasound probe 10. The pulse generation circuit repeatedly generates rate pulses for forming ultrasound transmitted at a given pulse repetition frequency (PRF). The PRF is also referred to as the rate frequency. The delay circuit focuses the ultrasound generated from the ultrasound probe 10 into a beam gives, to each rate pulse, a delay for each piezoelectric transducer element necessary to determine the transmission directivity. The trigger generation circuit applies a drive signal (drive pulse) to the ultrasound probe 10 at a timing based on each rate pulse that is given with a delay by the delay circuit. The delay corresponding to the transmission directivity is stored in the storage unit 180 and the delay circuit gives a delay with reference to the storage unit 180.

The transmitter/receiver 110 includes an amplifier circuit, an analog/digital (A/D) converter, and an adder and performs various types of processing on the reflected-wave signals that are received by the ultrasound probe 10 to generate, for example, radio frequency (RF) signals as reflected-wave data. The amplifier circuit amplifies the reflected-wave signals on a channel-by-channel basis. The A/D converter performs A/D conversion on the amplified reflected-wave signals and gives a delay time necessary to determine a receiving directionality. The adder performs an add operation on the reflected-wave signals given with the delay to generate reflected-wave data. The add operation of the adder enhances the reflected components from the direction corresponding to the reflected-wave signal receiving directionality and synthetic beams of ultrasound transmitting/receiving are formed in accordance with the receiving directionality and transmitting directionality. The delay corresponding to the receiving direction is stored in the storage unit 180. The reflected-wave data can be referred to as the “received signals” below.

The transmitter/receiver 110 has a function of changing delay information, transmission frequency, transmission drive voltage, the number of aperture elements instantly according to an instruction from the controller 190. Particularly, the transmission drive voltage is changed by using a linear amplifier transmitting circuit that can switch the value instantly or a mechanism for electrically switching between multiple power units. In this manner, the transmitter/receiver 110 controls the transmitting directionality and receiving directionality of ultrasound transmitting/receiving.

The signal processor 115 includes the B-mode processing unit 120 and the Doppler processing unit 130. The B-mode processing unit 120 receives reflected-wave data from the transmitter/receiver 110 and performs logarithmic amplification, envelope detection process, etc. on the reflected-wave data to generate data (B-mode data) expressing the signal intensity by luminance intensity. B-mode data is data where the luminance corresponding to the signal intensity is allocated to each sample point on scanning lines. The B-mode processing unit 120 is able to perform signal processing for performing harmonic imaging where harmonic components are visualized.

For example, contrast harmonic imaging (CHI) and tissue harmonic imaging (THI) are known for harmonic imaging. Furthermore, for harmonic imaging scanning methods, are known amplitude modulation (AM), phase modulation (PM), and amplitude-phase modulation (AMPM) with which effects of both AM and PM are obtained by combining AM and PM.

When performing CHI that is a visualization method for generating a contrast-enhanced image, the transmitter/receiver 110 transmits different waveforms several times in each ultrasound scanning line. For example, when performing CHI by AM, the transmitter/receiver 110 transmits, in a second time, a waveform whose polarity is corresponding to the same phase polarity of the waveform transmitted in a first time and amplitude ratio is corresponding to 1/2 of the amplitude ratio of the waveform transmitted in the first time, and generates each set of reflected-wave data. In other words, the transmitter/receiver 110 transmits, at least once in each scanning line, a first ultrasound pulse and a second ultrasound pulse, whose amplitude is equivalent to amplitude which is acquired by modulating amplitude of the first ultrasound pulse by using a given ratio, and acquires a received-signal group consisting of multiple received signals based on the transmitting. When performing harmonic imaging, the transmitter/receiver 110 transmits ultrasound by a scan sequence that is set by the controller 190, which will be described below. In AM, the B-mode processing unit 120 receives two sets of reflected-wave data on the patient P injected with microbubbles from the transmitter/receiver 110. The B-mode processing unit 120 corrects the ratio of the amplitude of the two sets of reflected-wave data, which are received from the transmitter/receiver 110, and calculates a difference between the two sets of reflected-wave data to generate reflected-wave data where the basic wave components are reduced and harmonic components (non-linear components) are extracted. In AM, for example, three types of ultrasound whose amplitude is at a ratio of “1:2:1” at the same phase polarity may be transmitted in each scanning line and three sets of reflected-wave data may be received. In such AM, the sets of reflected-wave data corresponding to the ultrasound of the amplitude ratio 1 are summed and a difference between the resultant reflected-wave data and reflected-wave data corresponding to the ultrasound of the amplitude ratio 2 is calculated to generate reflected-wave data where harmonic components (non-linear components) are extracted.

Furthermore, for example, when performing CHI by AMPM, the transmitter/receiver 110 transmits, in a second time, a waveform whose polarity is corresponding to the inverted phase polarity of the waveform transmitted in a first time and amplitude ratio is corresponding to 1/2 of the amplitude ratio of the waveform transmitted in the first time, and generates each set of reflected-wave data. In other words, the transmitter/receiver 110 transmits, at least once in each scanning line, a first ultrasound pulse and a second ultrasound pulse, whose phase polarity is equivalent to phase polarity which is acquired by inverting phase polarity of the first ultrasound pulse and amplitude is equivalent to amplitude which is acquired by modulating the amplitude of the first ultrasound pulse by using the given ratio, and acquires a received-signal group consisting of multiple received signals based on the transmitting. The second ultrasound pulse is an ultrasound pulse at an inverted phase polarity with respect to the phase polarity of the first ultrasound pulse, which is a second ultrasound pulse obtained by modulating the amplitude of the first ultrasound pulse such that the amplitude of the first ultrasound pulse and the amplitude of the second ultrasound pulse are at a given ratio. In the above-described AMPM, the B-mode processing unit 120 receives two sets of reflected-wave data on the patient P injected with microbubbles from the transmitter/receiver 110. The B-mode processing unit 120 corrects the ratio of amplitude of the two sets of reflected-wave data, which are received from the transmitter/receiver 110, and then sums the two set of reflected-wave data to generate reflected-wave data where the basic wave components are reduced and harmonic components (non-linear components) are extracted. In AMPM, for example, three types of ultrasound where the ultrasound transmitted for the second time has an opposite polarity with respect to the polarity of the ultrasound transmitted for the first time and the third time and whose amplitude is at a ratio of “1:2:1” may be transmitted in each scanning line and three sets of reflected-wave data then received may be summed to generate reflected-wave data where harmonic components (non-linear components) are extracted.

Subsequently, the B-mode processing unit 120 performs an envelope detection process etc. on the reflected-wave data where harmonic components are detected to generate B-mode data for generating a contrast-enhanced image. Accordingly, the image generator 140, which will be described below, can generate a contrast-enhanced image where the contrast agent that is flowing though the patient P is visualized and a tissue image where tissue is visualized.

The Doppler processing unit 130 performs frequency analysis on the velocity information from the reflected-wave data received from the transmitter/receiver 110, extracts the blood flow, tissue, and contrast-agent echo components by Doppler effect, and calculates blood flow information (Doppler data), such as the velocity average, dispersion, power etc., with respect to many points.

The image generator 140 generates, from the B-mode data generated by the B-mode processing unit 120, a B-mode image where the signal intensity is expressed by luminance intensity and generates, from the blood flow information generated by the Doppler processing unit 130, a color Doppler image where power components etc. representing the blood flow velocity, dispersion, and amount of blood flow etc. are displayed such that they can be identified by color. The data before being input to the image generator 140 can be referred to as the “raw data”.

Specifically, the image generator 140 removes noise components from the ultrasound scanning line signal row by performing a filtering process on the B-mode data and the Doppler data and stores the filtered data in the image memory 150. The image generator 140 then converts the ultrasound scanning line signal row of the filtered data into scanning line signal row in a normal video format for, for example, TV. The image generator 140 performs processing for adjusting the luminance and contrast, image processing such as spatial filtering, or combining processing for combining character information on various setting parameters, memories, etc. and outputs the processed signals to the monitor 30 as video signals. Accordingly, the ultrasound images, such as cross-sectional images that are generated by the image generator 140 and that represent the patient tissue shape, are displayed on the monitor 30.

The image memory 150 is a memory that stores ultrasound images that are generated by the image generator 140 and images that are generated by performing image processing on ultrasound images. For example, after a diagnosis, the operator can access images that are recorded during the examination in the image memory 150 and images can be reproduced as still images or as a video image using multiple images. The image memory 150 stores image luminance signals that have passed through the transmitter/receiver 110, other raw data, and images that are acquired via network etc., as required.

The software storage unit 160 is a storage area in which various apparatus control programs are loaded by the controller 190, which will be described below.

The interface unit 170 is an interface between the input device 20, external devices (not shown), and the network. Data, such as ultrasound images acquired by the ultrasound diagnosis apparatus 1, can be transferred by the interface unit 170 to other devices via the network.

The storage unit 180 stores various data groups of, for example, scan sequences, various apparatus control programs for performing image processing and image display processing etc., diagnostic information (e.g. patient IDs and doctor's opinions), diagnosis protocols, and various types of setting information. Various apparatus control programs may include a program in which the procedure for performing the same processing as that performed by the controller 190 is described. The storage unit 180 is also, as required, used to store ultrasound images that the image memory 150 stores. Various types of data stored in the storage unit 180 can be transferred to external devices via the interface unit 170.

The controller 190 is a control processor (central processing unit (CPU)) that implements the functions of an information processing device (computer) and controls whole processing performed by the ultrasound diagnosis apparatus 1. Specifically, the controller 190 loads various types of instructions and setting instructions that are input from the operator via the input device 20 and various types of apparatus control programs that are read from the storage unit 180 into the software storage unit 160 and, on the basis of various types of setting information, controls processing performed by the transmitter/receiver 110, the B-mode processing unit 120, the Doppler processing unit 130, and the image generator 140 and controls the monitor 30 to display ultrasound images stored in the image memory 150.

The whole configuration of the ultrasound diagnosis apparatus 1 according to the first embodiment has been described. The ultrasound diagnosis apparatus 1 according to the first embodiment having such a configuration generates, by, for example, performing CHI by AM to the patient P injected with microbubbles, a contrast-enhanced image where non-linearly components derived from contrast agent are further enhanced. However, depending on how the ultrasound diagnosis apparatus 1 is implemented (e.g., the system configuration, the aperture control and amplitude control) and the effect of nonlinearity of the circuit, the ultrasound diagnosis apparatus 1 is not necessarily able to completely cancel out linearly signals derived from tissue. In such a case, tissue-derived linearly signals remain, which reduces the bubble-tissue ratio in the contrast-enhanced images.

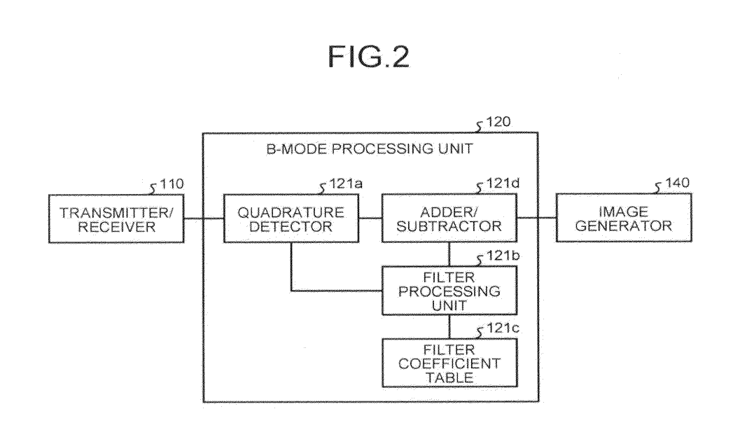

For this reason, the ultrasound diagnosis apparatus 1 according to the first embodiment performs waveform shaping on the reflected-wave data received from the transmitter/receiver 110 before performing beam addition/subtraction operations according to the modulation. With reference to FIGS. 2 to 6, the B-mode processing unit 120 according to the first embodiment will be described more in detail.

FIG. 2 is a block diagram of an exemplary configuration of the B-mode processing unit 120 according to the first embodiment. As shown in FIG. 2, the B-mode processing unit 120 includes a quadrature detector 121a, a filter processing unit 121b, a filter coefficient table 121c, and an adder/subtractor 121d. For the purpose of illustration, a case will be assumed where, when performing CHI by AM, the ultrasound diagnosis apparatus 1 transmits, twice and for each line, ultrasound that is modulated at an amplitude ratio of “1:2”, e.g., (0.5, 1.0), at the same phase polarity. The ultrasound of the amplitude ratio 2 is referred to as the high amplitude transmission rate and the ultrasound of the amplitude ratio 1 is referred to as the low amplitude transmission rate.

The quadrature detector 121a performs quadrature detection for converting RF signals that are output as reflected-wave data from the transmitter/receiver 110 into in-phase signals (I signal) and quadrature-phase signals (Q signals) of the base band. The quadrature detector 121a outputs the I and Q signals (hereinafter, referred to as the IQ signals) as reflected-wave data (received signals) to a downstream processor. In the first embodiment, the quadrature detector 121a outputs IQ signals at the low amplitude transmission rate to the adder/subtractor 121d and outputs IQ signals at the high amplitude transmission rate to the filter processing unit 121b. The IQ signals are one type of reflected-wave data (received signals).

The filter processing unit 121b includes a filter (not shown). The filter according to the first embodiment is a finite impulse response (FIR) filter. In the first embodiment, because filter processing is performed on the IQ signals, a complex FIR filter is used. Filter coefficients are set in the filter.

The filter coefficients are designed so as to minimize the energy of a non-contrast composite signal that is obtained by combining, by addition/subtraction operations according to the modulation, i.e., AM or AMPM, multiple non-contrast received signals that are multiple received signals of each ultrasound that is transmitted for multiple times for the same scanning line according to the modulation when there is not any contrast agent. The non-contrast received signals are signals received when there is not any contrast agent. In other words, the non-contrast received signals are multiple received signals acquired by the transmitter/receiver 110 when there is not any contrast agent in the scanning area. The non-contrast received signals are also referred to as the first received signal group. Furthermore, multiple received signals that are acquired by the transmitter/receiver 110 when there is a contrast agent in the scanning area are also referred to as the second received signal group.

It is preferable that, for designing filter coefficients, each of the multiple non-contrast received signals comes from a non-saturated area where the signal level is not saturated. In other words, it is preferable that each of the multiple received signals acquired by the transmitter/receiver 110 when there is not any contrast agent comes from a non-saturated area where the signal level is not saturated. Furthermore, it is preferable that, for designing a filter coefficient, each of the multiple non-contrast received signals is a signal resulting from transmission of ultrasound at a sound pressure that reduces occurrence of non-linearly propagation. In other words, each of the multiple received signals that are acquired by the transmitter/receiver 110 when there is not any contrast agent is a signal resulting from transmission of ultrasound at a sound pressure that reduces occurrence of non-linearly propagation.

When contrast-enhanced imaging is performed, the filter processing unit 121b filters at least one of the multiple received signals of each ultrasound that is transmitted for multiple times for the same scanning line according to the modulation. In other words, the filter processing unit 121b performs filter processing by using a filter on at least one of the multiple received signals that are contained in the second received signal group. For example, the filter processing unit 121b performs convolution on an IQ signal at the high amplitude transmission rate. In other words, the filter processing unit 121b corrects the IQ signal at the high amplitude transmission rate by using a filter coefficient that is stored in the filter coefficient table 121c. In AMPM, the filter processing unit 121b also performs convolution on an IQ signal at the high amplitude transmission rate. The filter processing unit 121b outputs the corrected IQ signal to the adder/subtractor 121d. The filter processing unit 121b acquires, from the filter coefficient table 121c, filter coefficients corresponding to the transmission conditions under which the contrast-enhanced imaging is performed and sets the acquired filter coefficients for a filter.

The filter coefficient table 121c stores multiple filter coefficients that are previously designed for multiple transmission conditions (frequency, position in transmission focus depth direction, sound pressure, depth of display etc.). For example, the filter coefficient table 121c stores filter coefficients that minimize the energy, after addition/subtraction, of each set reflected-wave data received from the patient P into which not any contrast agent is applied. In other words, the filter coefficient table 121c stores filter coefficients that minimize tissue-derived linearly signals. The filter coefficient table 121c stores, for example, coefficients that are acquired from another ultrasound diagnosis apparatus. A method of designing a filter coefficient will be described below.

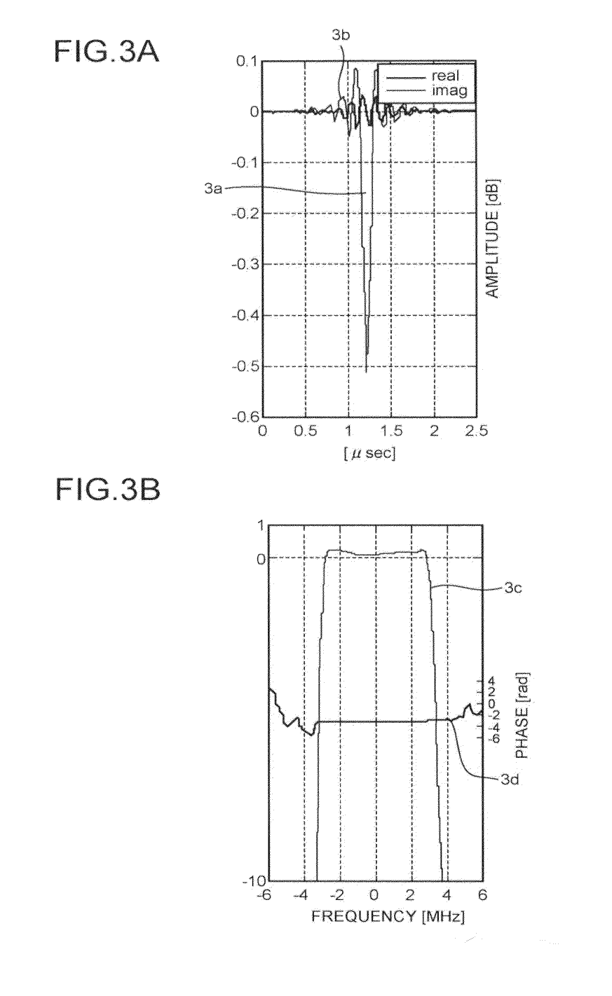

FIGS. 3A and 3B illustrate filter coefficients according to the first embodiment. FIGS. 3A and 3B illustrate exemplary coefficients and amplitude and phase property that are designed so as to minimize linear components remaining in a non-contrast composite signal that is acquired by AM without any non-contrast agent. FIGS. 3A and 3B illustrate exemplary filter coefficients and amplitude and phase property that are designed so as to perform filter processing on received signals (IQ signals) at the high amplitude rate to minimize the energy of a non-contrast composite signal. FIG. 3A illustrates exemplary filter coefficients represented by time components. The vertical axis in FIG. 3A represents the filter coefficient and the horizontal axis represents the time. The time represents the depth direction on the scanning line corresponding to the time after transmission of ultrasound. In FIG. 3A, 3a denotes the filter coefficient corresponding to the real part (I signal) of an IQ signal and 3b denotes the filter coefficient for the imaginary part (Q signal) of the IQ signal. As illustrated in FIG. 3A, each depth is associated with each filter coefficient. When performing filter processing in the time area, the filter processing unit 121b sequentially samples the waveform of the IQ signal for each depth and shapes the sampled IQ signal with the filter coefficient that is preset for each depth.

FIG. 3B is amplitude and phase property where the filter coefficients that are represented by time components in FIG. 3A are represented by frequency components by Fourier transformation. The horizontal axis in FIG. 3B represents the frequency and the vertical axis represents the amplitude characteristic or phase characteristic. In FIG. 3B, 3c denotes the amplitude characteristic and 3d denotes the phase characteristic. As illustrated in FIG. 3B, each frequency is associated with the amplitude characteristic and the phase characteristic. When performing filter processing in the frequency area, the filter processing unit 121b sequentially samples the waveform of the IQ signal for each frequency and shapes the waveform with the amplitude characteristic and phase characteristic that are previously set for each frequency. The amplitude characteristics of filter coefficient illustrated in FIG. 3B forms a concave shape in the visualized frequency band and, in the filter processing, the amplitude of the IQ signal is changed so as to cancel out linear components. The filter processing unit 121b performs filter processing on any one of the frequency area and the time area.

The adder/subtractor 121d outputs a composite signal that is obtained by, by addition/subtraction according to the modulation, combining multiple received signals that has been filtered by the filter processing unit 121b. For example, when performing CHI by AM, the adder/subtractor 121d generates a composite signal by summing the IQ signal at the low amplitude transmission rate that is input by the quadrature detector 121a and the corrected IQ signal at the high amplitude transmission rate that is input by the filter processing unit 121b. As described below, the amplitude ratio and phase polarity of two sets of reflected-wave data are taken into consideration for the filter coefficients and accordingly, when performing CHI by AM, the adder/subtractor 121d sums two sets of reflected-wave data to generate a composite signal. Furthermore, for example, when performing CHI by AMPM, the adder/subtractor 121d generates a composite signal by summing an IQ signal at the low amplitude transmission rate that is input by the quadrature detector 121a and a corrected IQ signal at the high amplitude transmission rate that is input by the filter processing unit 121b. Accordingly, the adder/subtractor 121d can cancel out the linearly signals derived from tissue and extract the harmonic components derived from the contrast agent. The composite signal generated by the adder/subtractor 121d serves as “B-mode data” for generating a contrast-enhanced image.

The B-mode processing unit 120 acquires a second composite signal by combining, on the basis of coefficients to reduce the energy of a first composite signal that is obtained by combining, in accordance with a given ratio, multiple received signals contained in a first received signal group that is acquired by the transmitter/receiver 110, multiple received signals contained in a second received signal group that is acquired by the transmitter/receiver 110 and that is different from the first received signal group. The image generator 140 generates an ultrasound image based on the second composite signal. In other words, the image generator 140 generates B-mode image data (contrast-enhanced image data) from the B-mode data and displays a contrast-enhanced image on the monitor 30.

The method of designing a filter coefficient will be described here. Filter coefficients according to the first embodiment are designed by a different ultrasound diagnosis apparatus by using another patient in which there is not any contrast agent, a patient P in which there is a contrast agent, and a phantom. In other words, filter coefficients according to the first embodiment are designed by using multiple non-contrast received signals that are signals received from a phantom or a living body. Each of the multiple received signals acquired by the transmitter/receiver 110 when there is not any contrast agent is a signal received from a phantom or a living body. The different ultrasound diagnosis apparatus has the same configuration as that of the ultrasound diagnosis apparatus 1 except that the B-mode processing unit of the different ultrasound diagnosis apparatus has a different configuration from that of the ultrasound diagnosis apparatus 1. Specifically, the different ultrasound diagnosis apparatus includes a filter coefficient designing unit in the B-mode processing unit. The filter coefficient processing performed by the filter coefficient designing unit will be described below. Filter coefficients may be designed by a work station.

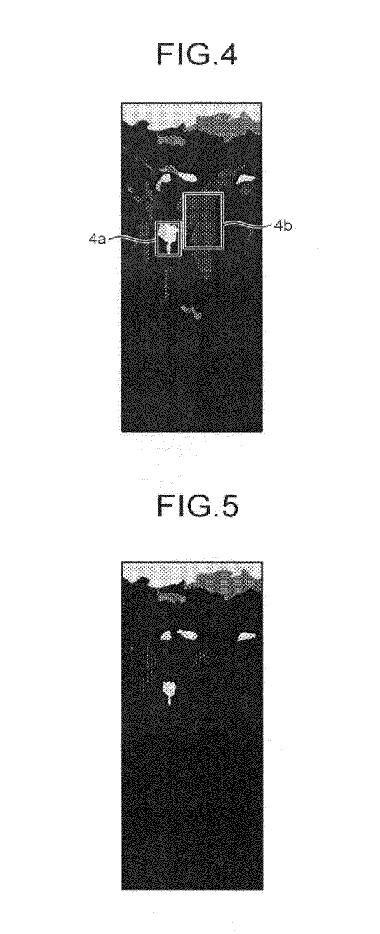

FIG. 4 illustrates the filter coefficient designing processing according to the first embodiment. FIG. 4 illustrates exemplary visualization of IQ signals of transmission signals of a high amplitude transmission rate. In FIG. 4, 4a denotes an exemplary area where the IQ signal of transmission signals at a high amplitude transmission rate is saturated and 4b denotes an exemplary area where the IQ signal of transmission signals at a high amplitude transmission rate is not saturated but tissue-derived linearly signals are generated (also referred to as the non-saturated area). To design filter coefficients, it is necessary to sample reflected-wave data avoiding the signal area of the contrast agent and a part where the signal level is saturated. For this reason, the filter coefficient designing unit according to the first embodiment receives a setting of a non-saturated area made by the operator. For example, the filter coefficient designing unit receives an instruction made by the operator for setting a rectangular area denoted by 4b shown in FIG. 4 for an area to be sampled. The filter coefficient designing unit designs a filter that minimizes the signal energy after addition of beam without any contrast agent.

Filter designing performed by the filter coefficient designing unit will be described below. For the purpose of illustration, exemplary filter designing in a case where CHI is performed by AM in which the transmission rate of transmission signals is 2. The filter coefficients form a filter that improves only the performance of canceling out linear components. Fort this reason, the filter coefficient designing unit sets a non-contrast component influence filter.

A transmission signal at the low amplitude transmission rate and a transmission signal at the high amplitude transmission rate, which are transmitted by the transmitter/receiver 110, are denoted by s_{TxL}(t) and s_{TxH}(t), respectively. A received signal (RF signal or IQ signal) corresponding to the transmission signal at the low amplitude transmission rate and a received signal (RF signal or IQ signal) corresponding to the transmission signal at the high amplitude transmission rate, which are acquired when those transmission signals are reflected by an object (reflection coefficient: ρ(z=2tc₀) where c₀ denotes the propagation velocity), are denoted by r_{TxL}(t) and r_{TxH}(t), respectively. Furthermore, the noise (white noise) that is received when the transmission signal at the low amplitude transmission rate is transmitted is denoted by n_{TxL}(t) and the noise (white noise) that is received when the transmission signal at the high amplitude transmission rate is transmitted is denoted by n_{TxH}(t), where “t” denotes the time and the position of each sample point along the depth direction is denoted by “t”.

The composite signal (also referred to as the AM signal) r_{AM}(t) that is obtained by summation after filter processing is represented by Equation (1):

$${r}_{AM} ( t ) = {r}_{T \times L} ( t ) + h ( t ) ⊗ {r}_{T \times H} ( t ) = \{ {s}_{T \times L} ( t ) + h ( t ) ⊗ {s}_{T \times H} ( t ) \} ⊗ ρ ( z = 2 {tc}_{0} ) + {n}_{T \times L} ( t ) + h ( t ) ⊗ {n}_{T \times H} ( t ) \qquad ( 1 )$$

where h(t) denotes a filter impulse response function. For h(t), the amplitude ratio and phase polarity of two sets of reflected-wave data are taken into consideration.

By performing Fourier transformation on the expressions on both sides of Equation (1) into frequency components, is obtained Equation (2):

$${R}_{AM} ( ω ) = {R}_{T \times L} ( ω ) + H ( ω ) {R}_{T \times H} ( ω ) = \{ {S}_{T \times L} ( ω ) + H ( ω ) {S}_{T \times H} ( ω ) \} P ( ω ) + {N}_{T \times L} ( ω ) + H ( ω ) {N}_{T \times H} ( ω ) \qquad ( 2 )$$

where are used the following Fourier transformation pairs:

r_{AM}(t)∞R_{AM}(ω), r_{TxL}(t) [symbol] R_{TxL}(ω), r_{TxH}(t) [symbol] R_{TxH}(ω)

h(t) [symbol] H(ω)

s_{TxL}(t) [symbol] S_{TxL}(ω), s_{TxH}(t) [symbol] S_{TxH}(ω)

ρ(z=2tc₀) [symbol] P(ω)

n_{TxL}(t) [symbol] N_{TxL}(ω), n_{TxH}(t) [symbol] N_{TxH}(ω)

The composite signal after the summation contains the residual signal and noise. For this reason, the filter coefficient designing unit calculates a filter H(ω) that minimizes the energy of the composite signal after summation. Equation (3) represents δ(ω) denoting the ensemble average of the absolute value square of the AM signal R_{AM} after summation. Equation (4) represents the nature of white noise. Equation (5) represents the nature of scattering coefficient, where ω denotes frequency.

$$\begin{matrix} \begin{matrix}ɛ ( ω ) = E [ {\vert {R}_{AM} ( ω ) \vert}^{2} ] \\ = E [ {\vert {R}_{T \times L} ( ω ) + H ( ω ) {R}_{T \times H} ( ω ) \vert}^{2} ] \\ = E [ {\vert \begin{matrix}\{ {S}_{T \times L} ( ω ) + H ( ω ) {S}_{T \times H} ( ω ) \} P ( ω ) + \\ {N}_{T \times L} ( ω ) + H ( ω ) {N}_{T \times H} ( ω )\end{matrix} \vert}^{2} ]\end{matrix} & ( 3 ) \\ \begin{matrix}E [ {N}_{T \times L} ( ω ) ] = E [ {N}_{T \times H} ( ω ) ] = 0 & Average \text{:} 0 \\ E [ {\vert {N}_{T \times L} ( ω ) \vert}^{2} ] = E [ {\vert {N}_{T \times H} ( ω ) \vert}^{2} ] = {σ}_{N}^{2} & Dispersion \text{:} {σ}_{N}^{2} \\ E [ {N}_{T \times L}^{*} ( ω ) {N}_{T \times H} ( ω ) ] = 0 & Independent ( not correlated )\end{matrix} \} & ( 4 )\end{matrix}$$

where * denotes a complex conjugate.

E[P(ω)]=0,E[|P(ω)|²]=σₚ² (5)

When not correlating to noise, ε(ω) is represented by Equation (6):

$$ɛ ( ω ) = [ {\vert {S}_{T \times H} ( ω ) \vert}^{2} {σ}_{P}^{2} + {σ}_{N}^{2} ] {\vert H ( ω ) + \frac{{S}_{T \times L} ( ω ) {S}_{T \times H}^{*} ( ω ) {σ}_{P}^{2}}{{\vert {S}_{T \times H} ( ω ) \vert}^{2} {σ}_{P}^{2} + {σ}_{N}^{2}} \vert}^{2} + 2 {σ}_{N}^{2} + \frac{{\vert {S}_{T \times L} ( ω ) \vert}^{2} {σ}_{P}^{2} + 2 {σ}_{N}^{2}}{{\vert {S}_{T \times H} ( ω ) \vert}^{2} {σ}_{P}^{2} + {σ}_{N}^{2}} {σ}_{N}^{2} \qquad ( 6 )$$

Because the second and following terms are constants in Equation (6), H(ω) for which ε(ω) is the minimum is represented by H_{Opt}(ω) for which the first term is the minimum as represented by Equation 7:

$${H}_{Opt} ( ω ) = - \frac{{S}_{T \times L} ( ω ) {S}_{T \times H}^{*} ( ω ) {σ}_{P}^{2}}{{\vert {S}_{T \times H} ( ω ) \vert}^{2} {σ}_{P}^{2} + {σ}_{N}^{2}} \qquad ( 7 )$$

If actual transmission spectra s_{TxL}(ω) and s_{TxH}(ω) are known, an optimal filter can be obtained in accordance with Equation (7). However, depending on the case, only reflection signals from a group of uniform scatterers may be acquired. In such a case, it is required to calculate a quasi-optimal filter. Ultrasound at a sound pressure that reduces occurrence of non-linearly propagation (e.g. ultrasound at a low/intermediate sound pressure) is transmitted and thus the received signals do not contain tissue-derived non-linear components.

Equation (8) represents a received signal r_{TxH}(t) that is obtained by transmitting a transmission signal at a high amplitude transmission rate. By performing Fourier transformation on the expressions on both sides of Equation (8), is obtained Equation (9).

r_{TxH}(t)=s_{TxH}(t) [symbol] ρ(z=2tc₀)+n_{TxH} (8)

R_{TxH}(ω)=S_{TxH}(ω)P(ω)+N_{TxH}(ω) (9)

By calculating the ensemble average of power spectrum of Equation (9), Equation (10) that is the denominator of Equation (7) is obtained.

$$\begin{matrix}E ⌊ {\vert {R}_{T \times H} ( ω ) \vert}^{2} ⌋ = E ⌊ {\vert {S}_{T \times H} ( ω ) P ( ω ) + {N}_{T \times H} ( ω ) \vert}^{2} ⌋ \\ = {\vert {S}_{T \times H} ( ω ) \vert}^{2} {σ}_{P}^{2} + {σ}_{N}^{2}\end{matrix} \qquad ( 10 )$$

Equation (11) represents a received signal r_{TXL}(t) that is obtained by transmitting a transmission signal at the low amplitude transmission rate. By performing Fourier transformation on the expressions on both sides of Equation (11), Equation (12) is obtained.

r_{TxL}(t)=s_{TxL}(t) [symbol] ρ(z=2tc₀)+n_{TxL} (11)

R_{TxL}(ω)=S_{TxL}(ω)P(ω)+N_{TxL}(ω) (12)

Equation (13) represents the product of the spectrum of the received signal obtained when a transmission signal at the high amplitude transmission rate is transmitted, i.e., the complex conjugate of Equation (9), and the spectrum of the received signal obtained when a transmission signal at the low amplitude transmission rate is transmitted, i.e., Equation (12). The ensemble average is represented by Equation (14) by using the nature represented by Equations (4) and (5).

R_{TxL}(ω)R_{TxH}*(ω)={S_{TxL}(ω)P(ω)+N_{TxL}(ω)}×{S_{TxH}*(ω)P*(ω)+N_{TxH}(ω)} (13)

E[R_{TxL}(ω)R_{TxH}*(ω)]=S_{TxL}(ω)S_{TxH}*(ω)σₚ² (14)

Equation (14) is the numerator of Equation (7). For this reason, by calculating each of the sample spectra R_{TxLi}(ω) and R_{TxHi}(ω) of the received signal corresponding to the transmission signal at the low amplitude transmission rate and the received signal corresponding to the transmission signal at the high amplitude transmission rate for one scanning line i, a filter coefficient can be given by Equation (15):

$$H ( ω ) = - \frac{{\sum }_{i} \{ {R}_{T \times L} ( ω ) {R}_{T \times H}^{*} ( ω ) \}}{{\sum }_{i} {\vert {R}_{T \times H} ( ω ) \vert}^{2}} \qquad ( 15 )$$

In this manner, the filter coefficient designing unit calculates a filter coefficient on the basis of the sample spectra R_{TxLi}(ω) and R_{TxHi}(ω) of the received signals of multiple scanning lines. In other words, as Equation (15) represents, a robust filter can be generated by the scanning-direction ensemble average processing performed by the filter coefficient designing unit. Accordingly, the ultrasound diagnosis apparatus 1 can improve the robustness. The filter coefficient designing unit may design a filter coefficient by calculating a filter coefficient only for one scanning line.

FIG. 5 illustrates the filter coefficient designing processing performed by the filter coefficient designing unit according to the first embodiment. FIG. 5 illustrates an exemplary visualization of IQ signals of transmission signals at the high amplitude transmission rate in a case where the IQ signals are corrected with filter coefficients that are designed by the filter coefficient designing unit. The imaging area shown in FIG. 5 is the same as the imaging area shown in FIG. 4. As shown in FIG. 5, when IQ signals of transmission signals at the high amplitude transmission rate are corrected with the filter coefficients, the tissue-derived linearly signals denoted by 4a in FIG. 4 are canceled out.

The filter coefficients form the filter and the filter is designed to have a filter length (i.e., kernel length, filter coefficient length, or tap length) approximately twice as long as the pulse length of transmitted ultrasound. In other words, the filter coefficient designing unit designs a filter having a filter length approximately twice as long as the pulse length. Accordingly, if the top of the waveform of a received signal to be corrected is positioned at the center of the filter, the end of the waveform of the received signal to be corrected is within the filter. If the filter length is too long, the spatial resolution in the depth direction may be unnecessarily lost while tissue-derived linearly signals can be canceled out. If the filter length is too short, tissue-derived linearly signals cannot be canceled out. The filter coefficient designing unit designs filter coefficients for each of various transmission conditions.

The filter coefficient table 121c stores filter coefficients that are designed for each of various transmission conditions. When imaging is performed, the filter processing unit 121b acquires a filter coefficient that matches a transmission condition or a filter coefficient close to the transmission condition from the filter coefficient table 121c and sets the filter coefficients for a filter.

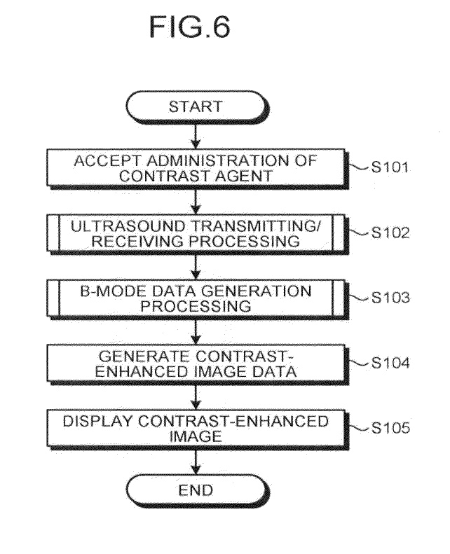

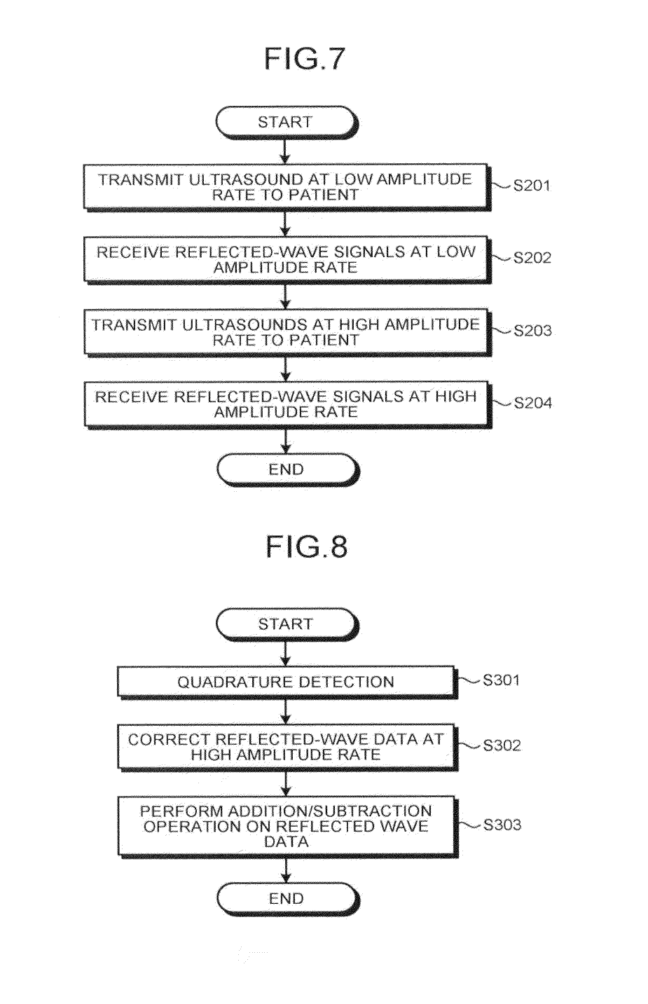

The procedure of processing performed by the ultrasound diagnosis apparatus 1 will be described with reference to FIGS. 6 to 8. FIG. 6 is a flowchart of the procedure of processing performed by the ultrasound diagnosis apparatus 1 according to the first embodiment. As shown in FIG. 6, the controller 190 accepts administration of a contrast agent (step S101). The transmitter/receiver 110 performs ultrasound transmitting/receiving processing (step S102). The ultrasound transmitting/receiving processing will be described in detail below with reference to FIG. 7.

The B-mode processing unit 120 performs B-mode data generation processing (step S103). The B-mode data generation processing will be described in detail below with reference to FIG. 8. The image generator 140 generates contrast-enhanced image data (step S104) and displays a contrast-enhanced image on the monitor 30 (step S105).

FIG. 7 is a flowchart of the procedure of ultrasound transmitting/receiving processing performed by the ultrasound diagnosis apparatus according to the first embodiment. The processing corresponds to the processing at step S102 shown in FIG. 6. As shown in FIG. 7, the transmitter/receiver 110 transmits ultrasound at a low amplitude transmission rate to a patient P (step S201). The transmitter/receiver 110 then receives reflected-wave signals at the low amplitude transmission rate and generates reflected-wave data (step S202). The transmitter/receiver 110 also transmits ultrasound at the high amplitude transmission rate to a patient P (step S203). The transmitter/receiver 110 then receives reflected-wave signals at the high amplitude transmission rate and generates reflected-wave data (step S204). The transmitter/receiver 110 may perform steps S203 and S204 first and then perform steps S201 and S202. The flowchart of FIG. 7 is performed on multiple scanning lines of one frame.

FIG. 8 is a flowchart of the procedure of the B-mode data generation processing performed by the ultrasound diagnosis apparatus 1 according to the first embodiment. The processing corresponds to the processing at step S103 shown in FIG. 6. The quadrature detector 121a of the B-mode processing unit 120 performs quadrature detection on the received signals that are input from the transmitter/receiver 110 (step S301). The quadrature detector 121a outputs reflected-wave data (IQ signal) at the low amplitude transmission rate to the adder/subtractor 121d and outputs reflected-wave data (IQ signal) at the high amplitude transmission rate to the filter processing unit 121b. The filter processing unit 121b reads filter coefficients that match or are close to transmission conditions from the filter coefficient table 121c to correct the reflected-wave data (IQ signal) at the high amplitude transmission rate (step S302). The adder/subtractor 121d performs an addition/subtraction operation on the reflected-wave data (IQ signal) at the low amplitude transmission rate and the corrected reflected-wave data (IQ signal) at the high amplitude transmission rate (step S303). The flowchart shown in FIG. 8 is performed on multiple scanning lines for one frame. Accordingly, B-mode data of contrast-enhanced imaging of one frame is generated.

As described above, the ultrasound diagnosis apparatus 1 according to the first embodiment includes a filter for which filter coefficients for canceling out tissue-derived linearly signals of received signals and, when performing CHI, processes at least one of the multiple received signals by using the filter. Accordingly, the ultrasound diagnosis apparatus 1 can cancel out tissue-derived linearly signals and extract contrast-agent-derived harmonic components. Accordingly, the ultrasound diagnosis apparatus 1 can generate a contrast-enhanced image offering a high bubble-tissue ratio.

Furthermore, each of the multiple received signals is a signal received from a phantom or a living body, i.e., received from a non-saturated area where the signal level is not saturated. Accordingly, the filter coefficient setting unit is able to design filter coefficients offering a high bubble-tissue ratio.

According to the first embodiment, filter coefficients are designed by using received signals resulting from transmission of ultrasound at a sound pressure that reduces occurrence of non-linearly propagation. Thus, according to the first embodiment, filter coefficients are designed under the condition that tissue-derived linear components are not contained, which makes it possible to design a filter that is able to cancel out tissue-derived linear components and extract only contrast-agent-derived harmonic components.

The filter coefficient table 121c stores multiple filter coefficients that are designed for each of multiple transmission conditions. Thus, an operator only needs to input a transmission condition for contrast-enhanced imaging in order for automatic selection of filter coefficients that matches the input transmission condition. Accordingly, a contrast-enhanced image offering a high bubble-tissue ratio can be generated easily.

### Second Embodiment

For the first embodiment, the case has been described where filter coefficients are previously set. However, in order to generate a contrast-enhanced image with further higher quality, it is more preferable to perform filter processing with filter coefficients that are adaptively designed according to the site to be imaged than to perform filter processing by using pre-designed filter coefficients. Thus, for the second embodiment, a case will be described where the ultrasound diagnosis apparatus 1 adaptively designs filter coefficients on the basis of received signals from a patient P to be scanned.

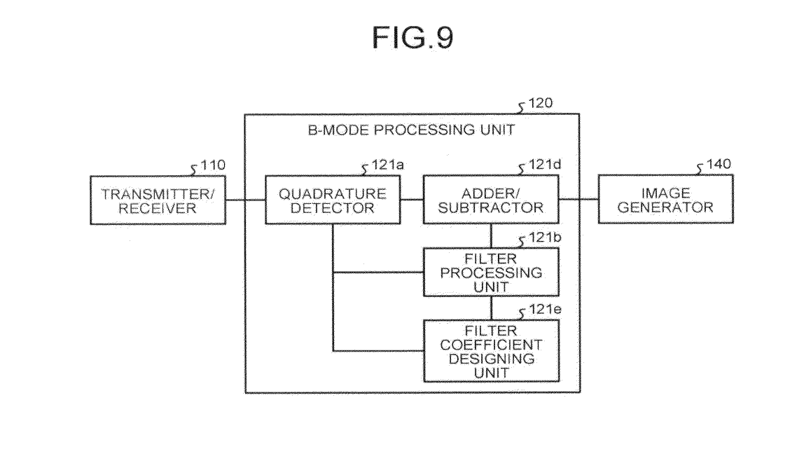

The ultrasound diagnosis apparatus 1 according to the second embodiment has the same configuration as that of the ultrasound diagnosis apparatus 1 shown in FIG. 1 according to the first embodiment except that the configuration of the B-mode processing unit of the ultrasound diagnosis apparatus 1 according to the second embodiment is partly different from that of the ultrasound diagnosis apparatus 1 according to the first embodiment. FIG. 9 is a block diagram of an exemplary configuration of the B-mode processing unit 120 according to the second embodiment. As illustrated in FIG. 9, the B-mode processing unit 120 according to the second embodiment includes the quadrature detector 121a, the filter processing unit 121b, the adder/subtractor 121d, and a filter coefficient designing unit 121e.

The filter coefficient designing unit 121e according to the second embodiment designs filter coefficients on the basis of multiple non-contrast received signals. In other words, the filter coefficient designing unit 121e according to the second embodiment designs filter coefficients on the basis of multiple received signals that are acquired by the transmitter/receiver 110 when there is not any contrast agent. The filter coefficient designing unit 121e according to the second embodiment has the same function as that of the filter coefficient designing unit of the first embodiment.

Each of multiple non-contrast received signals that are used by the filter coefficient designing unit 121e is a signal that is received under the same transmission conditions as those under which contrast-enhanced imaging is performed and received from a site to be imaged in the patient P in which contract-enhanced imaging is performed and there is not any contrast agent. Specifically, each of the multiple non-contrast received signals is a signal received from a site to be imaged to which not any contrast agent has reached or a site to be imaged before administration of a contrast agent. For example, the filter coefficient designing unit 121e designs filter coefficients in response to an input made by an operator. Specifically, before a contrast agent is administered, filter coefficients are designed at the timing when ultrasound scanning on a site to be imaged is started.

Furthermore, on the basis of the signal level of the multiple non-contrast received signals, the filter coefficient designing unit 121e determines whether the multiple received signals are from a non-saturated area. For example, on the basis of the energy and dynamic range of the received signals that are input, the filter coefficient designing unit 121e determines whether the received signals are saturated. Specifically, when a received signal whose energy is greater than the dynamic range is input, the filter coefficient designing unit 121e determines that the received signal is saturated. When designing filter coefficients, the filter coefficient designing unit 121e uses, for example, received signals at a high amplitude transmission rate to determine whether the received signals are saturated. The filter coefficient designing unit 121e may use, for example, received signals at a low amplitude transmission rate to determine whether the received signals are saturated. Alternatively, the filter coefficient designing unit 121e may use, for example, received signals at the low amplitude transmission rate and received signals at the high amplitude transmission rate to determine whether the received signals are saturated.

The filter coefficient designing unit 121e may design filter coefficients for a frame by using all scanning lines of the frame, design filter coefficients for a frame by using a part of multiple scanning lines of the frame, or design filter coefficients for a frame by using one scanning line of the frame. The filter coefficient designing unit 121e may receive a setting of a region of interest (ROI) made by an operator to design filter coefficients for a ROI by using all scanning lines of the ROI, design filter coefficients for a ROI by using a part of multiple scanning lines of the ROI, or design filter coefficients for a ROI by using one scanning line in the ROI. Such processing may be performed for multiple frames.

The filter processing unit 121b according to the second embodiment sets, for a filter, the filter coefficients that are designed by the filter coefficient designing unit 121e. The filter processing unit 121b then filters at least one of multiple received signals. In other words, the filter processing unit 121b according to the second embodiment performs filter processing on at least one of multiple received signals that are contained in a second received signal group. The adder/subtractor 121d according to the second embodiment outputs a composite signal (B-mode data) that is obtained by combining the filtered multiple received signals by addition/subtraction according to the modulation. The image generator 140 generates, from the B-mode data, a B-mode image where the signal intensity is represented by luminance intensity.

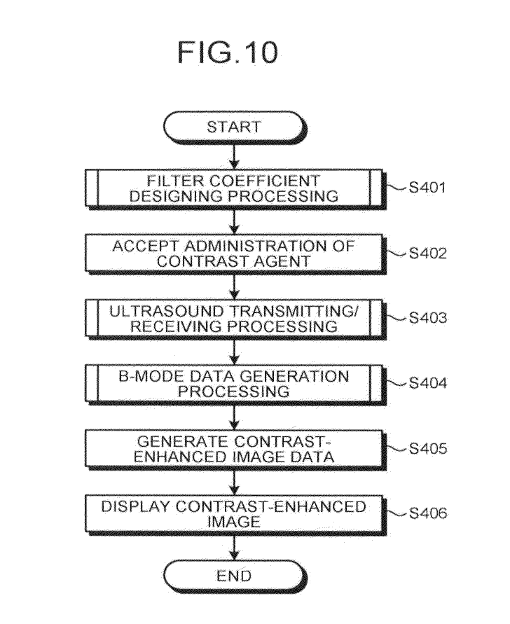

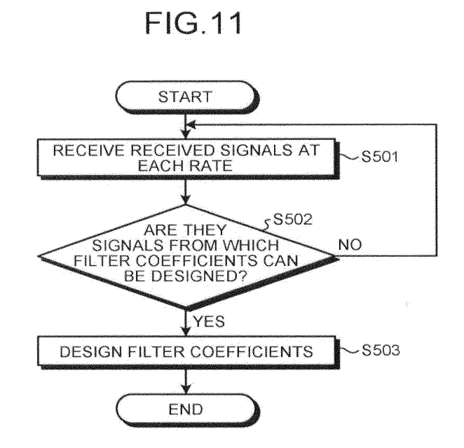

FIG. 10 is a flowchart of a procedure of processing performed by the ultrasound diagnosis apparatus 1 according to the second embodiment. As illustrated in FIG. 10, the filter coefficient designing unit 121e performs filter setting processing (step S401). The filter setting processing will be described in detail below with reference to FIG. 11. The controller 190 accepts administration of a contrast agent (step S402). The transmitter/receiver 110 performs ultrasound transmitting/receiving processing (step S403). The procedure of the ultrasound transmitting/receiving processing is the same as the procedure shown in FIG. 7.

The B-mode processing unit 120 then performs the B-mode data generation processing (step S404). The procedure of the B-mode data generation processing is the same as that of the procedure illustrated in FIG. 8. The image generator 140 generates contrast-enhanced image data (step S405) and displays a contrast-enhanced image on the monitor 30 (step S406).

After the administered contrast agent has flown out, the ultrasound diagnosis apparatus 1 may administer a contrast agent again and, when performing contrast-enhanced imaging under different transmission conditions, go to step S401 to repeat the processing at step S401 and the following steps.

FIG. 11 is a flowchart of a procedure of filter designing processing performed by the ultrasound diagnosis apparatus 1 according to the second embodiment. As illustrated in FIG. 11, the filter coefficient designing unit 121e receives reflected-wave data at each rate (step S501). For example, the filter coefficient designing unit 121e receives reflected-wave data at a low amplitude transmission rate and reflected-wave data at a high amplitude transmission rate.

The filter coefficient designing unit 121e determines whether they are signals from which filter coefficients can be designed (step S502). When the filter coefficient designing unit 121e does not determine that they are signals from which filter coefficients can be designed (NO at step S502), the processing proceeds to step S501 to receive reflected-wave data at each rate. When the filter coefficient designing unit 121e does not determine that they are signals from which filter coefficients can be designed, for example, the filter coefficient designing unit 121e may make a notification to the controller 190 to display, on the monitor, an instruction to change the probe contact position and an instruction to change the transmission conditions. Accordingly, it is determined whether the signals that are received again are signals from which filter coefficients can be designed.

In contrast, when the filter coefficient designing unit 121e determines that the signals that are received again are signals from which filter coefficients can be designed (YES at step S502), the filter coefficient designing unit 121e designs filter coefficients (step S503). For example, the filter coefficient designing unit 121e calculates filter coefficients from the reflected-wave data of transmission signals (IQ signal) at a low amplitude transmission rate and the reflected-wave data of transmission signals (IQ signal) at a high amplitude transmission rate. The filter coefficient designing unit 121e notifies the filter processing unit 121b of the calculated filter coefficients.

As describe above, the ultrasound diagnosis apparatus 1 according to the second embodiment designs filter coefficients adaptive to the patient P and transmission conditions. Each of the multiple non-contrast received signals is a signal received under the same transmission conditions as those under which contrast-enhanced imaging is performed and received from a site to be imaged in a patient in which contrast-enhanced imaging is performed and there is not any contrast agent. Accordingly, the ultrasound diagnosis apparatus 1 can generate a contrast-enhanced image offering a high bubble-tissue ratio.

It takes about 20 to 30 seconds for a contrast agent to reach the site to be imaged after the contrast agent is administered. For this reason, in the second embodiment, filter coefficients may be designed, not before but after the contrast agent is applied, by using the time until the contrast agent reaches the site to be imaged. In such a case, the processing uses the contrast agent administration timer that is normally used for contrast-enhanced imaging. The controller 190 acquires the time elapsing after administration of the contrast agent from the contrast agent administration timer. For example, an instruction for, after 10 minutes from administration of a contrast agent, automatically designing filter coefficients from a non-contrast received signal group acquired after administration of the contrast agent is transmitted to the filter coefficient designing unit 121e. In other words, the filter coefficient designing unit 121e designs filter coefficients in tandem with the contrast agent application timer that measures the time of administration of a contrast agent. Accordingly, the operator does not have to input an instruction for designing filter coefficients before a contrast agent is applied and filter coefficients can be designed automatically.

Furthermore, in the second embodiment, the B-mode processing unit 120 may include the filter coefficient table 121c. In such a case, the filter coefficient designing unit 121e stores adaptively-designed filter coefficients in the filter coefficient table 121c. Accordingly, when the filter coefficient table 121c stores filter coefficients that are designed from the same patient and under the same conditions, the filter processing unit 121b can read filter coefficients from the filter coefficient table 121c and set the filter coefficients for a filter.

What described in the first embodiment can be applied to the second embodiment except for that adaptive filter coefficients are designed by using a non-contrast received signal group that is acquired under conditions used for contrast-enhanced imaging (imaging conditions) before the contrast agent reaches.

### Third Embodiment

In the second embodiment, an example has been described where a filter is adaptively designed on the basis of signals received from a patient P to be scanned. There is a case where it is desired to acquire a contrast-enhance image under changed transmission conditions after administration of a contrast agent. In such a case, it is preferable to design filter coefficients that matches the changed transmission conditions. However, contrast agent bubbles remain in the patient P because it is after administration of the contrast agent and, for this reason, it is not possible to set filter coefficients. For this reason, in a third embodiment, the ultrasound diagnosis apparatus 1 designs a filter that can cancel out tissue-derived linearly signals in a state where there is not any contrast agent bubbles because of transmission of ultrasound to break down residual contrast agent bubbles. Ultrasound at a sound pressure that can break down a contrast agent is referred to as “flash”.

Upon receiving an operator's selecting of a “flash button”, the controller 190 according to the third embodiment issues, to the transmitter/receiver 110, an instruction for transmitting ultrasound (flash) at a sound pressure that can break down the contrast agent.

The filter coefficient designing unit 121e according to the third embodiment has the following functions in addition to the same functions as those of the filter coefficient designing unit 121e according to the second embodiment. For example, the filter coefficient designing unit 121e according to the third embodiment designs filter coefficients by using each of multiple non-contrast received signals that are signals received from a site to be imaged to which ultrasound at a sound pressure that can break off the contrast agent administration of the contrast agent has been transmitted.

The filter processing unit 121b according to the third embodiment sets filter coefficients that are designed by the filter coefficient designing unit 121e for a filter. The filter processing unit 121b filters at least one of multiple received signals as in the case of the second embodiment. As in the case of the second embodiment, the adder/subtractor 121d outputs a composite signal (B-mode data) that is obtained by combining filtered multiple received signals by addition/subtraction according to the modulation. The image generator 140 then generates, from the B-mode data, a B-mode image where the signal intensity is expressed by luminance intensity.

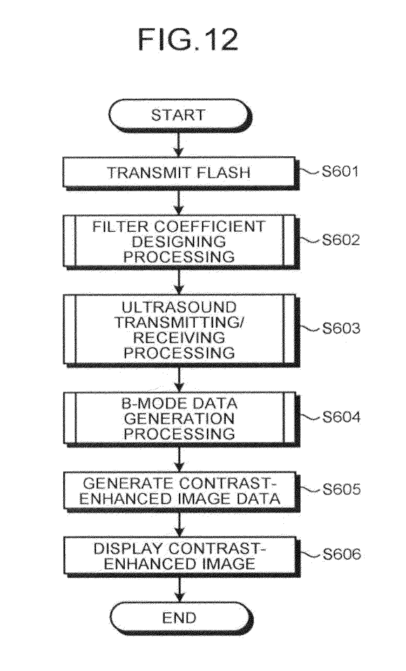

FIG. 12 is a flowchart of a procedure of processing performed by the ultrasound diagnosis apparatus 1 according to the third embodiment. As shown in FIG. 12, the controller 190 transmits flash for pre-set frames (step S601). The filter coefficient designing unit 121e then performs filter designing processing (step S602). The procedure of the filter designing processing is the same as the procedure shown in FIG. 11.

The transmitter/receiver 110 performs ultrasound transmitting/receiving processing (step S603). The procedure of the ultrasound transmitting/receiving processing is the same as the procedure shown in FIG. 7. The B-mode processing unit 120 then performs the B-mode data generation processing (step S604). The procedure of the B-mode data generation processing is the same as the procedure shown in FIG. 8. The image generator 140 generates a contrast-enhanced image data (step S605) and displays a contrast-enhanced image on the monitor 30 (step S606). When further performing contrast-enhanced imaging under the changed transmission conditions, the ultrasound diagnosis apparatus 1 proceeds to step S601 and performs processing to transmit flash and design filter coefficients. After designing filter coefficients, the ultrasound diagnosis apparatus 1 performs step S602 and the following steps.

As described above, by transmitting ultrasound that breaks off the contrast agent bubbles, the ultrasound diagnosis apparatus 1 according to the third embodiment can design adaptive filter coefficients even when the transmission conditions for contrast-enhanced imaging are changed as required. Thus, in the third embodiment, a contrast-enhanced image offering a high bubble-tissue ratio can be generated even when the transmission conditions for contrast-enhanced imaging are changed.

What described in the first and second embodiments can be applicable to the third embodiment except for that filter coefficients are re-designed by using transmission of flash.

### OTHER EMBODIMENTS

The embodiments have been described using examples where CHI is performed by AM. Alternatively, CHI may be performed by AMPM. Furthermore, THI may be performed by AMPM.

The embodiments have been described where the filter processing unit 121b filters IQ signals at a high amplitude transmission rate. Alternatively, for example, the filter processing unit 121b may filter the IQ signals at a low amplitude transmission rate. Alternatively, the filter processing unit 121b may filter IQ signals at a high amplitude transmission rate and IQ signals at a low amplitude transmission rate.

The embodiments have been described where the filter processing unit 121b filters IQ signals on which quadrature detection has been performed. Alternatively, for example, the filter processing unit 121b may filter RF signals. In other words, the filter processing unit 121b may filter IQ signals or RF signals. A complex FIR (finite impulse response) filter is used to process IQ signals and a real number FIR (finite impulse response) filter is used to process RF signals. In other words, when the second received signal group consists of IQ signals, the filter for which filter coefficients are set is a complex finite impulse response filter and, when the second received signal group consists of RF signals, the filter for which filter coefficients are set is a real number finite impulse response filter.

The filter coefficient designing unit 121e may calculate filter coefficients not only for one image (frame) but also two or more frames and design coefficients each of which is obtained by averaging the filter coefficients in each scanning line. The filter coefficient designing unit 121e may divide one frame into strip areas and set filter coefficients to each of the divided areas. The filter coefficient designing unit 121e may design filter coefficients according to the depth direction. Accordingly, the ultrasound diagnosis apparatus 1 can cancel out tissue-derived linearly signals when performing multi-focus and thus generates a contrast-enhanced image offering a high bubble-tissue ratio. The filter coefficient designing unit 121e may divide an area to be imaged into meshes and calculate filter coefficients for each of multiple pixels contained in the divided area.

According to at least one of the above-described embodiments, a contrast-enhanced image offering a high bubble-tissue ratio can be generated.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus comprising:
a transmitter/receiver configured to transmit, at least once in each scanning line, a first ultrasound pulse and a second ultrasound pulse, whose amplitude is equivalent to amplitude which is acquired by modulating amplitude of the first ultrasound pulse by using a given ratio, and acquire a received-signal group consisting of multiple received signals based on the transmitting;
a signal processor configured to, on the basis of a coefficient that reduces the energy of a first composite signal that is obtained by combining, in accordance with the given ratio, multiple received signals contained in a first received signal group acquired by the transmitter/receiver, acquire a second composite signal by combining multiple received signals contained in a second received signal group that is acquired by the transmitter/receiver and that is different from the first received signal group; and
an image generator configured to generate an ultrasound image based on the second composite signal.

2. The ultrasound diagnosis apparatus according to claim 1, wherein the second ultrasound pulse is an ultrasound pulse, whose phase polarity is equivalent to phase polarity which is acquired by inverting phase polarity of the first ultrasound pulse and amplitude is equivalent to amplitude which is acquired by modulating the amplitude of the first ultrasound pulse by using the given ratio.

3. The ultrasound diagnosis apparatus according to claim 1, wherein
the first received signal group consists of multiple received signals that are acquired by the transmitter/receiver when there is not any contrast agent in a scanning area, and
the second received signal group consists of multiple received signals that are acquired by the transmitter/receiver when there is a contrast agent in a scanning area.

4. The ultrasound diagnosis apparatus according to claim 2, wherein
the first received signal group consists of multiple received signals that are acquired by the transmitter/receiver when there is not any contrast agent in a scanning area, and
the second received signal group consists of multiple received signals that are acquired by the transmitter/receiver when there is a contrast agent in a scanning area.

5. The ultrasound diagnosis apparatus according to claim 1, wherein each of the multiple received signals that are acquired by the transmitter/receiver when there is not any contrast agent is a signal from a non-saturated area where the signal level is not saturated.

6. The ultrasound diagnosis apparatus according to claim 2, wherein each of the multiple received signals that are acquired by the transmitter/receiver when there is not any contrast agent is a signal from a non-saturated area where the signal level is not saturated.

7. The ultrasound diagnosis apparatus according to claim 1, wherein each of the multiple received signals that are acquired by the transmitter/receiver when there is not any contrast agent is a signal resulting from transmission of ultrasound at a sound pressure that reduces occurrence of non-linearly propagation.

8. The ultrasound diagnosis apparatus according to claim 2, wherein each of the multiple received signals that are acquired by the transmitter/receiver when there is not any contrast agent is a signal resulting from transmission of ultrasound at a sound pressure that reduces occurrence of non-linearly propagation.

9. The ultrasound diagnosis apparatus according to claim 1, wherein each of the multiple received signals that are acquired by the transmitter/receiver when there is not any contrast agent is a signal that is received from a phantom or a living body.

10. The ultrasound diagnosis apparatus according to claim 2, wherein each of the multiple received signals that are acquired by the transmitter/receiver when there is not any contrast agent is a signal that is received from a phantom or a living body.

11. The ultrasound diagnosis apparatus according to claim 1, wherein the coefficient forms a filter and a length of the filter is twice as long as a pulse length of ultrasound that is transmitted.

12. The ultrasound diagnosis apparatus according to claim 2, wherein the coefficient forms a filter and a length of the filter is twice as long as a pulse length of ultrasound that is transmitted.

13. The ultrasound diagnosis apparatus according to claim 1, wherein the signal processor includes:
a coefficient table configured to store a multiple coefficients that are previously designed for each of multiple transmission conditions; and
a filter processing unit configured to acquire, from the coefficient table, a coefficient corresponding to a transmission condition under which contrast-enhanced imaging is performed, set the acquired coefficient for a filter, and perform filter processing by using the filter on at least one of the received signals contained in the second received signal group.

14. The ultrasound diagnosis apparatus according to claim 2, wherein the signal processor includes:
a coefficient table configured to store a multiple coefficients that are previously designed for each of multiple transmission conditions; and
a filter processing unit configured to acquire, from the coefficient table, a coefficient corresponding to a transmission condition under which contrast-enhanced imaging is performed, set the acquired coefficient for a filter, and perform filter processing by using the filter on at least one of the received signals contained in the second received signal group.

15. The ultrasound diagnosis apparatus according to claim 1, wherein the signal processor includes:
a designing unit configured to design the coefficient on the basis of multiple received signals that are acquired by the transmitter/receiver when there is not any contrast agent; and
a filter processor configured to set, for a filter, the coefficient that is designed by the designing unit and perform filter processing, by using the filter, on at least one of the multiple received signals that are contained in the second received signal group.

16. The ultrasound diagnosis apparatus according to claim 2, wherein the signal processor includes:
a designing unit configured to design the coefficient on the basis of multiple received signals that are acquired by the transmitter/receiver when there is not any contrast agent; and
a filter processor configured to set, for a filter, the coefficient that is designed by the designing unit and perform filter processing, by using the filter, on at least one of the multiple received signals that are contained in the second received signal group.

17. The ultrasound diagnosis apparatus according to claim 1, wherein, when the second received signal group consists of IQ signals, the filter for which the coefficient is set is a complex finite impulse response filter and, when the second received signal group consists of RF signals, the filter for which the coefficient is set is a real number finite impulse response filter.

18. The ultrasound diagnosis apparatus according to claim 2, wherein, when the second received signal group consists of IQ signals, the filter for which the coefficient is set is a complex finite impulse response filter and, when the second received signal group consists of RF signals, the filter for which the coefficient is set is a real number finite impulse response filter.

19. A control method comprising:
transmitting, at least once in each scanning line, a first ultrasound pulse and a second ultrasound pulse, whose amplitude is equivalent to amplitude which is acquired by modulating amplitude of the first ultrasound pulse by using a given ratio, and acquiring a received-signal group consisting of multiple received signals based on the transmitting;
acquiring, on the basis of a coefficient that reduces the energy of a first composite signal that is obtained by combining, in accordance with the given ratio, multiple received signals contained in a first received signal group acquired by the transmitter/receiver, a second composite signal by combining multiple received signals contained in a second received signal group that is acquired by the transmitter/receiver and that is different from the first received signal group; and
generating an ultrasound image based on the second composite signal.

20. The control method according to claim 19, wherein the second ultrasound pulse, whose phase polarity is equivalent to phase polarity which is acquired by inverting phase polarity of the first ultrasound pulse and amplitude is equivalent to amplitude which is acquired by modulating the amplitude of the first ultrasound pulse by using the given ratio.
